(19) United States
(12) Patent Application Publication
(10) Pub. No.: US 2024/0342193 A1
(43) Pub. Date: Oct. 17, 2024

(54) Uses for testosterone

(71) Applicant: Menni Menashe Zinger, Ashkelon (IL)

(72) Inventor: Menni Menashe Zinger, Ashkelon (IL)

(21) Appl. No.: 18/135,256

(22) Filed: Apr. 17, 2023

(51) Int. Cl. A61K 31/568

(52) U.S. Cl. CPC A61K 31/568

(57) ABSTRACT

At this invention I present uses for testosterone at men mainly.

1. at conditions of very law testosterone at men the collagen tissues might dis apart for example at a 19 year old man that before that was a quiet great athlete and after possible poisoning with progesterone and lowering of testosterone levels because of that poisoning started to be un able to be active at sport and extreme effort become al most impossible. my assumption is that adding testosterone will improve his situation of stomach significantly.

2. at conditions of very law testosterone at men the urine bladder at 19 year old men that until that was with quiet normal ability to hold on urine even so not very good at that and after and after possible poisoning with progesterone and lowering of testosterone levels because of that poisoning started to suffer from urgency of need to urinate after drinking water or tea or coffee and need to urinate 20-25 times a day instead of average of 8-10 times a day at young men and my assumption is that law levels of testosterone at men cause this great need to urinate often. my assumption is that adding testosterone will improve his situation of stomach significantly.

3. at 20 years old man that was suppose poisoned at this age started problems and bowel stomach sounds and sensitivity to different food more than before, situation that continued 22 years until the age of 42 when there was a suspect additional poisoning there was a great fall at this man condition and the situation become un bearable al most and the stomach become very noisy and un rest at extreme way including puffed stomach that look like pregnant woman, condition that last already seven years. my assumption is that adding testosterone will improve his situation of stomach significantly.

1. using testosterone in order to treat damaged collagen tissues at men and woman and athletes and service people, by injecting, tablets, pills, gel, patch at excepted dosage or even high dosage than normal significantly until sage maximum dosage.

2. using testosterone in order to treat often need to urinate at man and maybe woman too with very law dosage of testosterone by injecting, tablets, pills, gel, patch at excepted dosage or even high dosage than normal significantly until sage maximum dosage.

3. using testosterone in order to treat un quiet stomach/irritable bowel sounds including at extreme way at men with too law testosterone and or high level of progesterone at men body, maybe also at woman with using very law dosage of testosterone by injecting, tablets, pills, gel, patch at excepted dosage or even high dosage than normal significantly until sage maximum dosage.
